(19) United States
(12) Patent Application Publication
(10) Pub. No.: US 2016/0361478 A1
(43) Pub. Date: Dec. 15, 2016

(54) DRESSING FOR NEGATIVE PRESSURE WOUND TREATMENT

(71) Applicant: Parasol Medical LLC, Buffalo Grove, IL (US)

(72) Inventor: Patrick E. Eddy, Allendale, MI (US)

(21) Appl. No.: 15/179,346

(22) Filed: Jun. 10, 2016

Related U.S. Application Data
(60) Provisional application No. 62/173,495, filed on Jun. 10, 2015.

(51) Int. Cl. A61M 1/00; A61F 13/00

(52) U.S. Cl. CPC A61M 1/0088; A61F 13/00017; A61F 13/00029; A61F 13/00063; A61F 13/00068; A61F 13/00995; A61M 1/0023; A61M 1/0086

(57) ABSTRACT

A wound dressing is provided for use in negative pressure wound therapy. The wound dressing comprises a cover and a wound dressing pad. The wound dressing pad comprises a foam pad and an antimicrobial substrate provided at the wound side of the foam pad. The wound dressing pad is configured for placement over a wound site with the antimicrobial substrate disposed between the foam pad and the wound site. The cover is disposed over the wound dressing pad to secure the wound dressing pad to the wound site.

### CROSS-REFERENCE TO RELATED APPLICATION

This patent application claims priority under 35 U.S.C. §119(e) upon U.S. Provisional Patent Application No. 62/173,495, entitled “DRESSING FOR NEGATIVE PRESSURE WOUND TREATMENT” filed on Jun. 10, 2015, by Patrick E. Eddy, the entire disclosure of which is incorporated herein by reference.

### BACKGROUND OF THE INVENTION

The present invention generally relates to a dressing used in the application of negative pressure wound therapy.

### SUMMARY OF THE INVENTION

According to an aspect of the present invention, a wound dressing is provided for use in negative pressure wound therapy. The wound dressing comprises a cover and a wound dressing pad. The wound dressing pad comprises a foam pad and an antimicrobial substrate provided at the wound side of the foam pad. The wound dressing pad is configured for placement over a wound site with the antimicrobial substrate disposed between the foam pad and the wound site. The cover is disposed over the wound dressing pad to secure the wound dressing pad to the wound site.

According to another aspect of the present invention, a system is provided for the treatment of wounds by applying a negative pressure to a wound site. The system comprises a regulated vacuum source and a wound dressing provided at the wound site and coupled to the regulated vacuum source. The wound dressing comprises a cover and a wound dressing pad. The wound dressing pad comprises a foam pad and an antimicrobial substrate provided at the wound side of the foam pad. The wound dressing pad is configured for placement over a wound site with the antimicrobial substrate disposed between the foam pad and the wound site. The cover is disposed over the wound dressing pad to secure the wound dressing pad to the wound site.

According to another aspect of the present invention, a method is provided for making a wound dressing for use in negative pressure wound therapy. The method comprises providing a cover and a wound dressing pad. The wound dressing pad comprises a foam pad and an antimicrobial substrate provided at the wound side of the foam pad. The wound dressing pad is configured for placement over a wound site with the antimicrobial substrate disposed between the foam pad and the wound site. The cover is disposed over the wound dressing pad to secure the wound dressing pad to the wound site.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is a side view of a wound dressing according to an embodiment of the present invention shown when applied to a wound;

FIG. 2 is a perspective view of the wound dressing of FIG. 1 configured for attachment to a patient's leg;

FIG. 3 is a side view of the attachment pad shown in FIG. 1;

FIG. 4 is a plan view of the bottom surface of the attachment pad shown in FIG. 1; and

FIG. 5 is a perspective view of a regulated vacuum source that may be used with the wound dressing shown in FIG. 1.

### DETAILED DESCRIPTION OF PREFERRED EMBODIMENTS

Reference will now be made in detail to the present preferred embodiments of the invention, examples of which are illustrated in the accompanying drawings. Wherever possible, the same reference numerals will be used throughout the drawings to refer to the same or like parts. In the drawings, the depicted structural elements are not to scale and certain components are enlarged relative to the other components for purposes of emphasis and understanding.

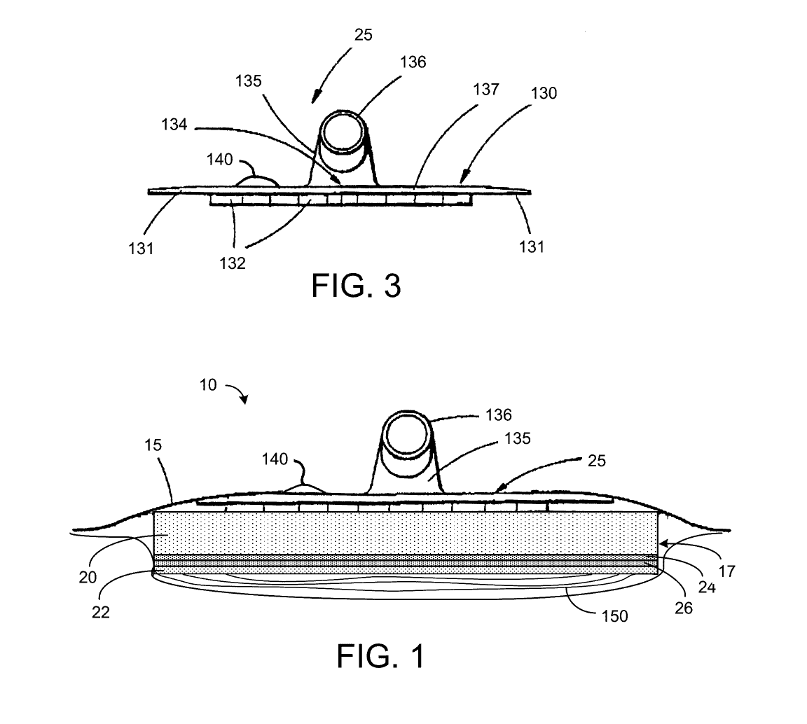

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations and step sequences, except where expressly specified to the contrary. It is also to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

Described herein are several embodiments of a wound dressing 10 (FIG. 1) for use in negative pressure wound therapy. Negative pressure wound therapy is a form of therapy whereby a partial vacuum (thus the phrase “negative pressure”) is applied to a wound 150. The partial vacuum on the wound 150 is thought to aid in healing of the wound 150. The wound dressing 10 is intended to be placed directly over the wound surface and used in conjunction with negative pressure wound therapy of the wound 150.

Wound treatment systems that treat a wound using a vacuum or negative pressure are known. Examples of such systems are disclosed in U.S. Pat. Nos. 4,382,441; 4,392,858; 4,655,754; 4,826,494; 4,969,880; 5,100,396; 5,261,893; 5,527,293; 5,636,643; 5,645,081; 6,071,267; 6,117,111; 6,135,116; 6,142,982; 6,174,306; 6,345,623; 6,398,767; 6,520,982; 6,553,998; 6,814,079; 7,198,046; and 7,216,651; and in U.S. Patent Application Publication No. US 2009/0043268 A1. These systems utilize either a manual pump or a portable vacuum pump to draw air and fluid from the wound site. The wound site is covered with a dressing. Typical wound dressings include a wound dressing pad such as a gauze or foam pad placed on the wound with a cover wrapped around the wound to hold the gauze or foam pad against the wound. The dressing further includes a tube connected to a vacuum source and to a connector that is disposed on the opposite side of the gauze or foam pad so as to extend through the cover such that a vacuum may be applied to the wound site.

In wound treatment systems such as the one described above, the wound dressing pad is sized and shaped to fit in and over the wound to be treated, and thus the wound dressing is in direct contact with the wound. In prior systems, a gauze or foam pad is used as the wound dressing pad so as to allow air to flow around the wound. The air flow is caused by the application of a vacuum. Because the vacuum also tends to draw fluids from the wound and through the wound dressing pad, the wound tissue can grow into the wound dressing pad or otherwise stick to the wound dressing pad. This causes problems in that the wound does not heal properly and can also reopen when the wound dressing is removed or changed. In addition, the removal of a wound dressing pad that is stuck to the wound can be particularly uncomfortable for the patient. These forms of dressings need to be changed frequently. Although foam pads do not need to be replaced as frequently as gauze, foam pads need to be changed every three days as the flesh forming over the healing wound tends to grow into the porous openings of the foam.

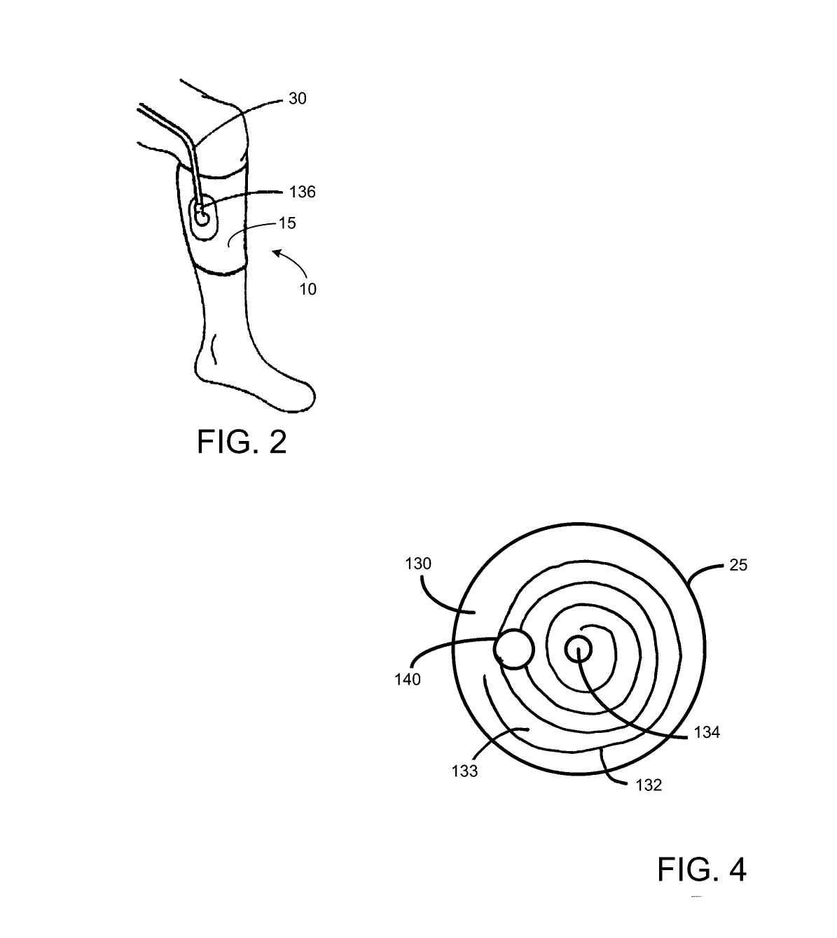

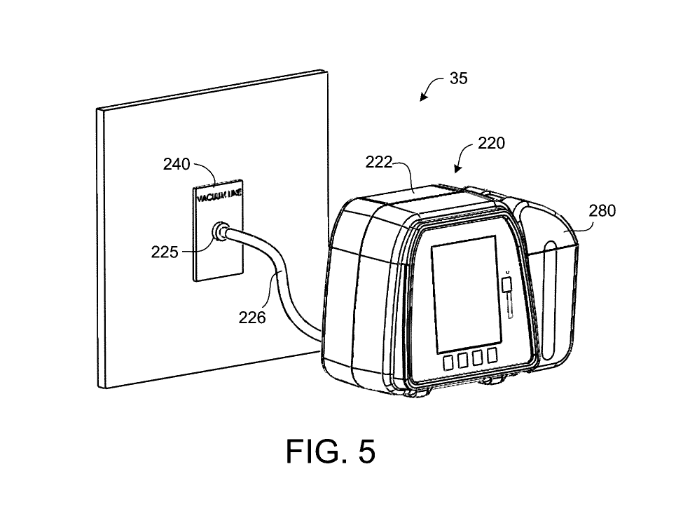

An example of the wound dressing 10 is shown in FIG. 1. As illustrated, wound dressing 10 includes a cover 15 (or drape), a wound dressing pad 17 including a foam pad 20 and an antimicrobial substrate 22 attached to the wound side of the foam pad 20. The wound dressing 10 may further include an attachment pad 25 for connecting to a tube 30 (FIG. 2), which in turn connects to a regulated vacuum source 35 (FIG. 5).

Cover 15 may be made of membrane permeable, semi-permeable or non-permeable materials that are commercially available, an example being material referred to as TAGODERM®, which is available from the 3M (Minnesota Mining and Manufacturing) Company of St. Paul, Minn. As shown in FIG. 2, cover 15 may wrap around a person's limb to secure the wound dressing 10 to the wound site.

Foam pad 20 may be a granufoam-urethane base or whitefoam-PVA base foam. An example of suitable foam is a granufoam available from Kinetic Concepts, Inc. (KCI) of San Antonio, Tex. The foam pad 20 may have an antimicrobial material integrated therein as disclosed in U.S. Patent Application Publication No. 2014/0271794 A1, the entire disclosure of which is incorporated herein by reference.

Antimicrobial substrate 22 may be a nylon fiber substrate coated with silver. More specifically, antimicrobial substrate 22 may be a SilverIon® antimicrobial negative pressure wound dressing available from Argentum Medical, LLC, which is a single layer of knitted continuous nylon fiber substrate composed of 73% 2/70/68 Unifi nylon+17% 4DG nylon+4% 1/40/13 nylon+6% 40-denier elastane that is circumferentially coated with metallic silver at the rate of 5.05 mg/cm² (50.5 g/m²). The silver surface coating is approximately 0.8-1.0 μm thick and contains approximately 1% silver oxide.

By utilizing antimicrobial substrate 22 on the wound side of foam pad 20, the flesh forming over the healing wound 150 will not grow into the porous openings of the foam pad and thus, the wound dressing 10 may be used for up to seven days, which is the typical maximum duration a patient would undergo negative pressure wound therapy.

Antimicrobial substrate 22 may be secured to foam pad 20 by an adhesive. Such an adhesive may be an apertured film 24 that may be laminated or co-extruded on foam pad 20. For example, such an aperture film may be Delnet® geometric apertured film, which is a lightweight, nonwoven netting fabric that, when co-extruded, may serve as an adhesive bonding layer. Delnet® geometric apertured films are available from DelStar Technologies, Inc. of Middletown, Del.

An optional layer of a carbon material 26 may be provided between foam pad 20 and antimicrobial substrate 22 to absorb odors. The carbon layer 26 may be provided on foam pad 20 or on antimicrobial substrate 22 or incorporated in the apertured film 24. Carbon layer 26 may also be separately provided and adhered using apertured film 24 on one side and an additional apertured film on the other side.

An attachment pad 25 such as those disclosed in U.S. Pat. Nos. 6,345,623; 6,553,998; and 6,814,079 or in U.S. Patent Application Publication No. US 2009/0043268 A1 may also be used with the inventive system. The entire disclosures of each of these documents are incorporated herein in their entireties. In addition, a TRACKPAD™ available from KCI may also be employed.

The attachment pad disclosed in U.S. Patent Application Publication No. US 2009/0043268 A1 includes a mechanical device to provide a visual acknowledgement of a predetermined level of vacuum at or near the wound site. In general, an attachment pad 25 such as that shown in FIGS. 3 and 4 comprises a flange portion 130 having a tapered edge 131, and a profile which may be of any desired shape. On the face of the flange 130 that is intended for contact with the foam pad 20 are one or more projections 132. The purpose of these projections is to provide one or more fluid channels 133 facilitating the flow of fluids from any point of the flange to a central aperture 134, from which it is intended to apply suction. The attachment pad 25 includes a connector 135, located above the aperture 134, having a tubular end 136 adapted for receiving and connecting to the vacuum conduit tube 30. The tubular end 136 may have an outwardly tapered portion to facilitate feeding tube 30 into the connector 25. The upper surface 137 of the attachment pad 25 has a substantially smooth surface with the exception of a bubble or dome 140 (described further below). A linear attachment tube may be used in lieu of the attachment pad.

In use, the connector 135 is sized so that it extends through an aperture in the wound cover 15, with the adhesive surface of cover 15 around the aperture bonded to the smooth surface 137 of the flange 130. The flange 130 of the attachment pad 25 may be circular as shown in FIG. 4. Alternatively, the flange may be any other shape.

FIG. 1 shows the attachment pad 25 attached at a wound site of a patient. The attachment pad 25 is pressed into firm contact with wound dressing pad 17, which is itself pressed into contact with a wound area 150. The attachment pad 25 and wound dressing pad 17 are pressed into contact with the wound area 150 by cover 15. The adhesive surface of cover 15 is bonded to the patient's skin outside the periphery of the wound dressing pad 17 and attachment pad 25. As noted above, it is also bonded to upper surface 137 of the attachment pad 25. As also mentioned above, an aperture is formed in the cover 15 to permit the connector portion 135 to extend upwardly through the drape.

As mentioned above, attachment pad 25 may have a convex bubble or dome 140 formed in one of its surfaces that is sucked inward with increasing vacuum pressure at or near the wound site 150. The size, thickness, and material used for the bubble or dome could be used to calculate an approximate vacuum indication that would be changeable in the mold itself. The attachment pad 25 could include multiple bubbles that each indicate different vacuum levels such as 50, 100, and 150 mm Hg.

As shown in FIG. 5, regulated vacuum source 35 may include an electronically controlled suction regulator 220 that connects to an external vacuum source 240. Such an external vacuum source may include the built-in central vacuum system of a healthcare facility, central vacuum pump remotely located from the suction regulator, or a separate portable vacuum pump. Such a connection may be via an appropriately configured coupler 225 and a supply hose 226. This system may be used for numerous health provider procedures and devices. As described further below, this system may have special safety features built in to protect the patient. The term “built-in” vacuum system is intended to refer to vacuum systems that are plumbed into the building structure of a healthcare facility and is not intended to cover a vacuum pump mounted to a wall or other structure of the patient's room. “Healthcare facility” is intended to include hospitals, outpatient treatment facilities, doctors' offices, nursing homes, and any other facility in which healthcare services are provided.

As shown in FIG. 5, regulated vacuum source 35 may include electronically controlled suction regulator 220 as a single unit having a housing 222 with integrated regulation of the vacuum and a containment apparatus 280 that will contain solids and liquids, but let gaseous materials pass to the atmosphere. The purpose is to provide a safe method of providing either a constant or an intermittent/modulated vacuum for a physician or health provider to use on a patient or to connect to a device that may or may not be used on a patient. This device is electronically controlled and will perform various functions including the ability to lock out users from changing settings to alarming functions for safety and efficacy.

Regulated vacuum source 35 may also be implemented using an integrated electronically controlled suction regulator 220 having an internal vacuum pump such that connection to a separate vacuum source is not required. Vacuum source 35 may alternatively be provided using a manual pump. An example of a portable manual pump that may be used with the dressing is disclosed in U.S. Provisional Application No. 62/331,668, entitled “PORTABLE PUMP FOR NEGATIVE PRESSURE WOUND THERAPY,” filed on May 4, 2016 by Patrick E. Eddy, the entire disclosure of which is incorporated herein by reference.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the claims as interpreted according to the principles of patent law, including the doctrine of equivalents.

What is claimed is:

1. A wound dressing for use in negative pressure wound therapy, comprising:
a cover; and
a wound dressing pad, said wound dressing pad comprising:
a foam pad; and
an antimicrobial substrate provided at the wound side of said foam pad,
wherein said wound dressing pad is configured for placement over a wound site with said antimicrobial substrate disposed between said foam pad and the wound site, and
wherein said cover is disposed over said wound dressing pad to secure said wound dressing pad to the wound site.

2. The wound dressing of claim 1, wherein said antimicrobial substrate comprises a nylon fiber substrate coated with silver.

3. The wound dressing of claim 1, wherein said foam pad comprises a granufoam having a urethane base.

4. The wound dressing of claim 1, wherein said wound dressing pad further comprises an adhesion layer disposed on said foam pad for securing said antimicrobial substrate to said foam pad.

5. The wound dressing of claim 4, wherein said adhesion layer comprises a nonwoven netting fabric co-extruded on a surface of said foam pad.

6. The wound dressing of claim 1 and further comprising an attachment pad for connecting the wound dressing to a vacuum source via a tube, wherein said wound dressing pad has a first surface for contacting the wound and a second surface opposite said first surface, and wherein said attachment pad disposed on said second surface of said wound dressing pad and wherein said attachment pad is partially covered by said cover.

7. The wound dressing of claim 1 and further comprising a carbon layer disposed between said foam pad and said antimicrobial substrate.

8. A system for the treatment of wounds by applying a negative pressure to a wound site, the system comprising:
a regulated vacuum source; and
the wound dressing of claim 1 provided at the wound site and coupled to said regulated vacuum source.

9. A system for the treatment of wounds by applying a negative pressure to a wound site, the system comprising:
a regulated vacuum source; and
a wound dressing provided at the wound site and coupled to said regulated vacuum source, said wound dressing comprising:
a cover; and
a wound dressing pad, said wound dressing pad comprising:
a foam pad; and
an antimicrobial substrate provided at the wound side of said foam pad,
wherein said wound dressing pad is configured for placement over a wound site with said antimicrobial substrate disposed between said foam pad and the wound site, and
wherein said cover is disposed over said wound dressing pad to secure said wound dressing pad to the wound site.

10. The system for the treatment of wounds of claim 9, wherein said antimicrobial substrate comprises a nylon fiber substrate coated with silver.

11. The system for the treatment of wounds of claim 9, wherein said foam pad comprises a granufoam having a urethane base.

12. The system for the treatment of wounds of claim 9, wherein said wound dressing pad further comprises an adhesion layer disposed on said foam pad for securing said antimicrobial substrate to said foam pad.

13. The system for the treatment of wounds of claim 12, wherein said adhesion layer comprises a nonwoven netting fabric co-extruded on a surface of said foam pad.

14. The system for the treatment of wounds of claim 9 and further comprising an attachment pad for connecting the wound dressing to a vacuum source via a tube, wherein said wound dressing pad has a first surface for contacting the wound and a second surface opposite said first surface, and wherein said attachment pad disposed on said second surface of said wound dressing pad and wherein said attachment pad is partially covered by said cover.

15. The system for the treatment of wounds of claim 9 and further comprising a carbon layer disposed between said foam pad and said antimicrobial substrate.

16. A method of making a wound dressing for use in negative pressure wound therapy, comprising:
providing a cover; and
providing a wound dressing pad comprising:
a foam pad; and
an antimicrobial substrate provided at the wound side of said foam pad,
wherein said wound dressing pad is configured for placement over a wound site with said antimicrobial substrate disposed between said foam pad and the wound site, and
wherein said cover is disposed over said wound dressing pad to secure said wound dressing pad to the wound site.

17. The method of claim 16, wherein said antimicrobial substrate comprises a nylon fiber substrate coated with silver.

18. The method of claim 16, wherein said foam pad comprises a granufoam having a urethane base.

19. The method of claim 16, and further comprising providing an adhesion layer on said foam pad for securing said antimicrobial substrate to said foam pad.

20. The method of claim 16 and further comprising providing a carbon layer between said foam pad and said antimicrobial substrate.
